(19) United States
(12) Patent Application Publication
(10) Pub. No.: US 2007/0167805 A1
(43) Pub. Date: Jul. 19, 2007

(54) Ultrasound Imaging

(76) Inventor: Gregory Thomas Clement, Medford, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/554,427

(22) Filed: Oct. 30, 2006

Related U.S. Application Data
(60) Provisional application No. 60/731,405, filed on Oct. 28, 2005.
(60) Provisional application No. 60/761,556, filed on Jan. 23, 2006.

(51) Int. Cl. A61B 8/14

(52) U.S. Cl. 600/459

(57) ABSTRACT

An ultrasound imaging system for use in producing an image of an object in a region of interest includes: an exciter configured to provide an excitation signal; a transducer coupled to the exciter and configured to produce, in response to the excitation signal, an ultrasound field whose complex frequency content varies with field location; a receiver configured to receive ultrasound signals reflected by the object and to produce indicia of the received reflected ultrasound signals; and a processor coupled to the receiver and configured to cross-correlate the indicia of the received reflected ultrasound signals with indicia of the ultrasound field at pixels in the region of interest to determine image pixel intensities of the region of interest for producing an image.

### CROSS-REFERENCE TO RELATED ACTIONS

This application claims the benefit of U.S. Provisional Application No. 60/731,405 filed Oct. 28, 2005 and U.S. Provisional Application No. 60/761,556 filed Jan. 23, 2006, which are incorporated herein by reference.

### STATEMENT AS TO FEDERALLY-SPONSORED RESEARCH

This invention was made at least in part with Government support under Grant No. NCI R21EB004353, awarded by the National Institutes of Health. The Government has certain rights in this invention.

### BACKGROUND

Ultrasound backscatter imaging is a well established modality that uses a combination of time-of-flight measurement and beam focusing to locate an object in space. Resolution along the ultrasound propagation axis is determined by the time duration of an impulsive signal, which is directly related to the signal bandwidth. Radial resolution is dictated by the ultrasound beamwidth, which is directly related to frequency. Thus, higher radial resolution images are created with higher source frequencies.

### SUMMARY

In general, in an aspect, the invention provides an ultrasound imaging system for use in producing an image of an object in a region of interest, the system including: an exciter configured to provide an excitation signal; a transducer coupled to the exciter and configured to produce, in response to the excitation signal, an ultrasound field whose complex frequency content varies with field location; a receiver configured to receive ultrasound signals reflected by the object and to produce indicia of the received reflected ultrasound signals; and a processor coupled to the receiver and configured to cross-correlate the indicia of the received reflected ultrasound signals with indicia of the ultrasound field at pixels in the region of interest to determine image pixel intensities of the region of interest for producing an image.

Implementations of the invention may include one or more of the following features. The transducer is configured to produce the ultrasound field such that the field has unique waveforms at each pixel location in the region of interest in the absence of the object, the waveforms being different in at least one of shape and timing relative to production of the ultrasound field. The pixels have a pitch of at least about ⅛ of a wavelength of a center frequency of the transducer. The transducer is configured to provide a frequency response that varies linearly along a length of an aperture of the transducer. The transducer and the receiver are each stationary relative to the object and provide a single imaging channel. The transducer is configured as a hexahedral right prism having two nonparallel surfaces, with one of the nonparallel surfaces being a radiating surface. The transducer is polarized normal to the radiating surface. The excitation signal is a spike. The receiver is separate from, and disposed in, the transducer. The receiver is configured as a point receiver. The transducer is configured to produce ultrasound signals with frequencies from about 200 KHz to at least about 2.5 MHz. The transducer is configured to produce ultrasound signals over a range of frequencies with a −6 dB bandwidth of between about 120% and about 166%. The system farther includes a display coupled to the processor, and the processor and the display are configured to produce a two-dimensional image of the region of interest from the image pixel intensities of the region of interest.

In general, in another aspect, the invention provides a method of imaging an object in a region of interest using ultrasound, the method including: producing an ultrasound field such that waveforms at centers of predetermined pixel locations in the region of interest in the absence of the object would be unique; receiving ultrasound signals reflected by the object; producing indicia of the received reflected ultrasound signals; cross-correlating the indicia of the received reflected ultrasound signals with indicia of the waveforms at pixels in the region of interest to determine image pixel intensities of the region of interest for producing an image; and producing an image of the object using the image pixel intensities.

Implementations of the invention may include one or more of the following features. Waveforms at different pixels are different in at least one of shape and timing relative to production of the ultrasound field. Producing the ultrasound field includes providing a frequency response at a transducer that varies linearly along a length of an aperture of the transducer. Producing the ultrasound field is performed at a transducer that is stationary relative to the object and receiving ultrasound signals reflected by the object is performed at a receiver that is stationary relative to the object. Producing the ultrasound field includes applying a spike excitation signal to a transducer. Producing the ultrasound field includes producing ultrasound signals with frequencies from about 200 KHz to at least about 2.5 MHz. Producing the ultrasound field includes producing ultrasound signals over a range of frequencies with a −6 dB bandwidth of between about 120% and about 166%.

In general, in another aspect, the invention provides an ultrasound transducer system including an air-backed hexahedral right prism transducer having first and second surfaces that are nonparallel with respect to each other, the transducer being configured to receive an excitation signal and to radiate, in response to the excitation signal, ultrasound waves from the first surface, the transducer being configured to radiate ultrasound waves along a length of the first surface and having frequencies in a range from a first frequency to a second frequency, the second frequency being higher than the first frequency, and the length of the first surface being at least about three times as long as a wavelength of the second frequency.

Implementations of the invention may include one or more of the following features. The transducer includes a piezo ceramic material. The transducer includes a composite material containing the piezo ceramic material. The length of the first surface is at least about five times as long as the wavelength of the second frequency. The length of the first surface is at least about ten times as long as the wavelength of the second frequency. The length of the first surface is at least about twenty times as long as the wavelength of the second frequency. The system further includes an exciter coupled to the transducer and configured to provide the excitation signal to the transducer, the excitation signal including a broadband spike.

In general, in another aspect, the invention provides an ultrasound transducer system including a single transducer configured to receive an excitation signal and to radiate, in response to the excitation signal, ultrasound waves along a length of an aperture with the ultrasound waves having frequencies in a range from a first frequency to a second frequency, the second frequency being higher than the first frequency, and the length of the aperture being at least about three times as long as a wavelength of the second frequency.

In accordance with implementations of the invention, one or more of the following capabilities may be provided. Imaging systems may be constructed to image in higher than three dimensions with reduced electronic and emitter complexity. Imaging can be performed at lower overall frequencies than with existing systems. Imaging can be performed more deeply and/or in higher-attenuating regions than with previous systems. Relatively high-resolution imaging can be achieved at lower frequencies than previously used for equal resolution imaging. Fine resolution imagery in highly attenuating tissues and in deep-set regions-of-interest can be achieved. Fine resolution imaging can be achieved at lower cost than previous systems.

These and other capabilities of the invention, along with the invention itself, will be more fully understood after a review of the following figures, detailed description, and claims.

### BRIEF DESCRIPTION OF THE FIGURES

FIG. 1 is a simulated sequence of a time-limited excitation signal (1A), a time-extended scattered signal (1B), and a cross-correlation of the two signals (1C).

FIG. 2 is a plot of contours of peak amplitude as a function of position in front of an emitting transducer.

FIG. 3 is a schematic diagram of a system for determining two-dimensional images using a single, stationary ultrasound transmitter.

FIG. 4 is a schematic diagram of a calibration setup of the system shown in FIG. 3.

FIG. 5 is a block flow diagram of a process performing calibration using the setup shown in FIG. 4.

FIG. 6 is a block flow diagram of a process of producing two-dimensional images of an object using the system shown in FIG. 3.

FIG. 7 is a plot of normalized pressure response of a first experimental transducer as obtained with impulse-reflection.

FIG. 8 shows radiated pressure magnitude and phase field plots of a second experimental transducer.

FIG. 9 shows images of three representative frequency-isolated pressure magnitude and phase fields of the impulse radiation of the second transducer.

FIG. 10 shows plots of an impulse response of the second transducer as measured by impulse-reflection (10A) and radiation force (10B).

FIG. 11 is a schematic diagram of a setup of the system shown in FIG. 3 using a steel wire target.

FIG. 12A is a plot of a simulation for two 0.25 mm scatterers, in the region of interest of the system shown in FIG. 3, placed with 1 mm separation orthogonal to the transducer surface.

FIG. 12B is an image of the region of interest shown in FIG. 12A.

FIG. 12C is a plot of a simulation for two 0.25 mm scatterers, in the region of interest of the system shown in FIG. 3, placed with (FIG. 12A) 1 mm separation parallel to the transducer surface.

FIG. 12D is an image of the region of interest shown in FIG. 12B.

FIG. 13 shows reconstructed images of cross-correlation fields for three placements of scattering targets using the first transducer as the excitation source and a 0.5-mm diameter pressure sensitive hydrophone as a detector.

FIG. 14 shows images of cross-correlation fields for four placements of scattering targets using the second transducer as the excitation source and a 2.0-mm diameter pressure sensitive hydrophone as the detector.

FIG. 15 shows images of cross-correlation fields for three placements of two scattering targets using the second transducer as the excitation source and a 2.0-mm diameter pressure sensitive hydrophone as the detector.

### DETAILED DESCRIPTION OF PREFERRED EMBODIMENTS

Embodiments of the invention provide techniques for backscatter imaging. For example, a two-dimensional B-mode imaging system includes a single transducer paired with a single point-like receiving element, providing a single imaging channel. The transducer geometry is that of a hexahedral right prism having two nonparallel surfaces, one of these being the radiating surface. The transducer is polarized normal to this surface and produces an acoustic filed with a large bandwidth and a radiation pattern whose complex frequency content varies with field location. The field produced, e.g., by an impulsive driving potential, preferably is not focused, not time-localized, does not contain a high center frequency, and has a complex spectral pattern with spatially dependent amplitude and phase spectrum over a region of interest (ROI), which can be used to reconstruct the location of scatters received by a broadband point detector. The system can use a reconstruction designed to resolve two-dimensional radial and axial information from the single stationary transmitter and the single receiver. Reflections from objects that may be within the ROI are recorded by the single, unfocused point-like receiver, and the ROI is reconstructed by interpretation of a single waveform. With this system, the acoustic field's frequency content is spatially dependent, providing spatial information of a signal recorded along a single channel in the time domain. This system is exemplary, however, and not limiting of the invention as other implementations in accordance with the disclosure are possible.

Numeric and experimental analysis evaluated techniques for providing a two-dimensional image from a single recorded time trace. A transducer design that produced a diffuse frequency-separated ultrasound field was used. The techniques leveraged a finding that applying acoustic scatter theory and the Born approximation, it can be shown that weak scatterers in a frequency-separated acoustic field will scatter linearly, retaining a unique complex frequency signature. The scattered field can be analyzed for both spectral and temporal content, revealing enough information to localize scatter sites in 3-dimensional space. With experimental transducer designs, target localization with single time traces was shown for single and multiple scatterers in 2-dimensional imaging fields as discussed below. It was found that target localization can be achieved with excitation wavelengths substantially larger than the individual scattering profiles.

### Theory

The Scattered Field

An ultrasound emitter is assumed that has a driven line source (e.g., continuously driven) that varies in frequency as a function of position ω(r). The contribution to the overall linear pressure field from an arbitrary point at r₀ on the source radiating into a homogeneous space is given by

$${p}_{ω} ( r , t ) = - ⅈ {c}_{0} {k}_{0} {ρ}_{0} {S}_{ω} {g}_{ω} ( {r}_{{s}_{ω}} \text{\vert} {r}_{0} ) \qquad ( 1 )$$

where S_{ω} is the source strength, c₀ the sound speed, ρ₀ the density, k₀=ω/c₀ and g_{ω} is equal to

$${g}_{ω} ( {r}_{{s}_{ω}} \text{\vert} {r}_{0} ) = \frac{{ⅇ}^{- ⅈ k \vert r - {r}_{0} \vert}}{4 π \vert r - {r}_{0} \vert} . \qquad ( 2 )$$

If this field, however, encounters an ROI of spatially varying density ρ and sound speed c, the time-harmonic acoustic pressure due to this point on the source may then be described by the wave equation

$$ρ ▽ \cdot ( \frac{1}{ρ} ▽ {p}_{ω} ) + \frac{{ω}^{2}}{{c}^{2}} {p}_{ω} = 0. \qquad ( 3 )$$

It is assumed that no scattering exists outside the ROI. To express the field in an integral form, Eq (3) is first multiplied by ρ₀/ρ and then −(∇²+ω²/c₀²)p_{ω} is added to both sides of the equation, giving the form of a harmonically driven distributed source,

$${▽}^{2} {p}_{ω} + {k}_{0}^{2} {p}_{ω} = ▽ \cdot ( \{ 1 - \frac{{ρ}_{0}}{ρ} \} ▽ {p}_{ω} ) + \{ {k}_{0}^{2} - \frac{{ρ}_{0}}{ρ} {k}^{2} \} {p}_{ω} , \qquad ( 4 )$$

which, in the absence of the scattering region, reduces to a Helmoltz equation describing p_{ω} in a sourceless medium. Equation (4) may be written in the form of a Lippmann-Schwinger integral equation

$${p}_{ω} ( {r}_{R} ) = - ⅈ {c}_{0} {k}_{0} {ρ}_{0} {S}_{ω} {g}_{ω} ( {r}_{{s}_{ω}} \text{\vert} {r}_{R} ) + {\int \int \int }_{ROI} ( ▽ \cdot ( {q}_{ρ} ( r ) ▽ {p}_{ω} ) + {q}_{κ} ( r ) {k}_{0}^{2} {p}_{ω} ) g ( r \text{\vert} {r}_{R} ) ⅆ V , \qquad ( 5 )$$

which represents the incident wave plus the scattered wave. The function q_{ρ}(r)=1−ρ₀/ρ provides a measure of the spatial variation in density while

$${q}_{κ} ( r ) = 1 - \frac{{ρ}_{0} {c}_{0}^{2}}{ρ {c}^{2}}$$

is a function of variation in compressibility. Further assuming that the scattered field is weak, such that the first order Born approximation holds, the scattered pressure recorded at a point receiver located at r_{R} is linearly dependent on the initial source function and Eq (5) becomes

$$p ( {r}_{R} ) \approx ⅈ {c}_{0} {k}_{0} {ρ}_{0} {S}_{ω} \times [ {g}_{ω} ( r \text{\vert} {r}_{0} ) + {\int \int \int }_{ROI} [ \begin{matrix} {g}_{ω} ( {r}_{R} \text{\vert} r ) ▽ \cdot ( {q}_{ρ} ( r ) ▽ {g}_{ω} ( r \text{\vert} {r}_{0} ) ) + \\ {q}_{κ} ( r ) {k}_{0}^{2} {g}_{ω} ( {r}_{R} \text{\vert} r ) {g}_{ω} ( r \text{\vert} {r}_{0} )\end{matrix} ] ⅆ V ] . \qquad ( 6 )$$

The second term in the integrand of Eq. (6) may be expanded using the standard vector identity:
φ(∇·A)=∇·(φA)−A·∇φ (7)
so that by the divergence theorem,
∫∫∫∇·(φA)dV=∫φA·dS, (8)
where S is the surface surrounding the ROI, the first term in the identity given by Eq. (7) integrates to zero. Equation (6) then becomes

$${p}_{ω} ( {r}_{R} ) \approx ⅈ {c}_{0} {k}_{0} {ρ}_{0} {S}_{ω} \times [ {g}_{ω} ( r \text{\vert} {r}_{0} ) + {\int \int \int }_{ROI} [ \begin{matrix} {q}_{ρ} ( r ) ▽ {g}_{ω} ( {r}_{R} \text{\vert} r ) ▽ {g}_{ω} ( r \text{\vert} {r}_{0} ) + \\ {q}_{κ} ( r ) {k}_{0}^{2} {g}_{ω} ( {r}_{R} \text{\vert} r ) {g}_{ω} ( r \text{\vert} {r}_{0} )\end{matrix} ] ⅆ V ] . \qquad ( 9 )$$

The point receiver (theoretically simultaneously) receives the integral sum of the pressure due to (theoretically all) points on the source r₀. The scattered acoustic pressure at r_{R} is time-dependent as described by

$$p ( {r}_{R} , t ) = {\sum }_{{r}_{0}} {\int \int \int }_{ROI} [ {q}_{ρ} ( r ) {P}_{ρ} ( {r}_{R} , r , {r}_{0} ) + {q}_{κ} ( r ) {P}_{κ} ( {r}_{R} , r , {r}_{0} ) ] {ⅇ}^{ⅈ ω ( {r}_{0} ) t} ⅆ V . \qquad ( 10 )$$

where the kernels P_{ρ}(r_{R}, r, r₀) and P_{κ}(r_{R}, r, r₀), having the dimension of pressure per unit volume, are obtained by combining terms in Eq. (9).
Image Reconstruction

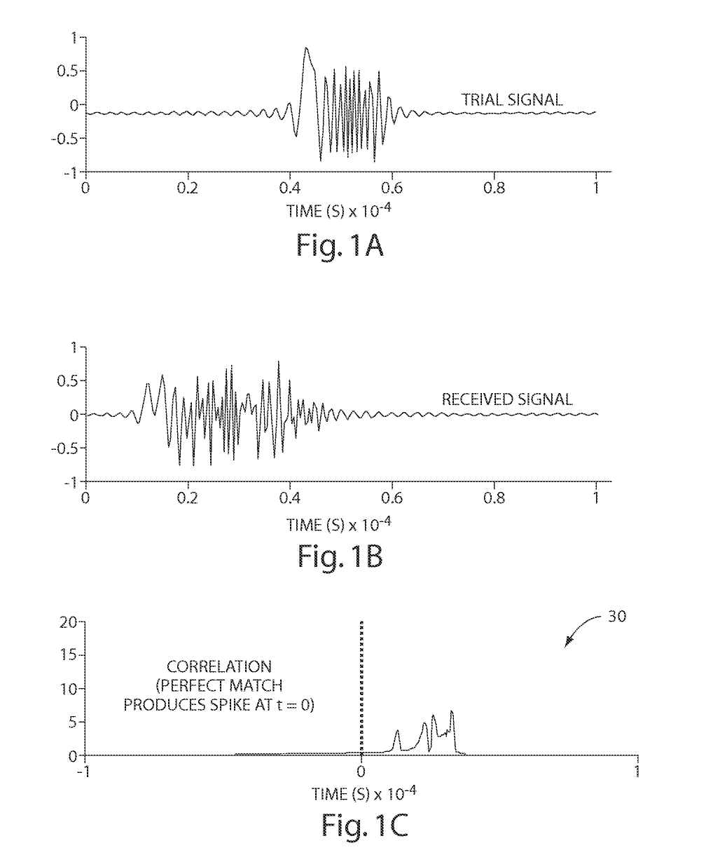

To reconstruct images, a search is performed for solutions of the scattering functions q_{ρ}(r) and q_{κ}(r) in Eq. (10), assuming that the field transmitted from the transducer, and thus P_{ρ}(r_{R}, r, r₀) and P_{κ}(r_{R}, r, r₀), are known. The transducer can be powered by an impulsive signal with a time duration much shorter (<0.1×) than that of the period of the highest radiating frequency and with a repetition frequency equal to or lower than that of the lowest radiating frequency. Even in the ideal case where the transducer is continuously driven, amplitude peaks produced by the source are time limited. A numeric example of such a signal is provided in FIG. 1.

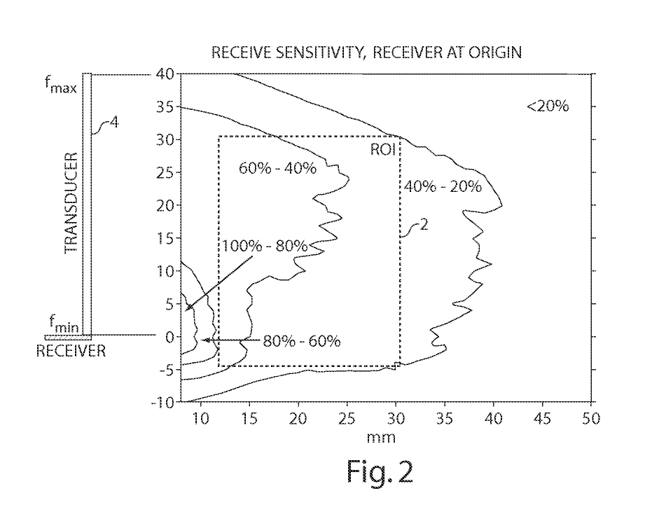

The field produced by the impulsively driven transducer is received by a point receiver. Due to the profile of the transmitted field and the spatial sensitivity of the receiver itself, the spatial sensitivity depends highly on the receiver's position relative to the transducer, as shown in FIG. 2. FIG. 2 shows a plot of contours of peak amplitude as a function of position in front of an emitting transducer, assuming equal-strength scattering at all points. Here the ROI is selected as a rectangular area with amplitude variation between 20% and 60% of the peak. A box 2 in front of a transducer 4 in FIG. 2 indicates the ROI, which has relatively flat sensitivity. If a scattering field were located within this region, a time-extended signal would be received, as illustrated in FIG. 1.

The single waveform received may be processed to solve for the qₛ. A high-resolution database of the signal P_{Q}(r_{R}, t) is developed as a function of position due to a single point scatterer Q(r) located within an otherwise homogeneous ROI. This response could be measured by scanning a point-like scatterer through the ROI or, alternatively, calculated for scatterers of varying density and compressibility. A cross-correlation between this signal and the expected response P_{Q}(r_{R}, t) is calculated for each point in the ROI according to:

$${I}_{Q ( r )} ( {t}^{′} , {r}_{R} ) = {\int }_{- ∞}^{∞} {p}_{Q ( r )} ( {r}_{R} , t ) p ( {r}_{R} , t + {t}^{′} ) ⅆ t . \qquad ( 11 )$$

By the property of the correlation integral, first order scattering produces a peak at t=0 provided that part of P_{Q}(r_{R}, t) is superimposed in p(r_{R}, t). Thus the cross-correlation at t=0 is selected as the image intensity strength at r. The process can be repeated for each position r over the ROI to form an image. The cross-correlation provides an inverse (or pseudo-inverse) operation representing the separation of a structural materials, underwater objects, and underground objects.n object function from the signal.

### Exemplary Systems

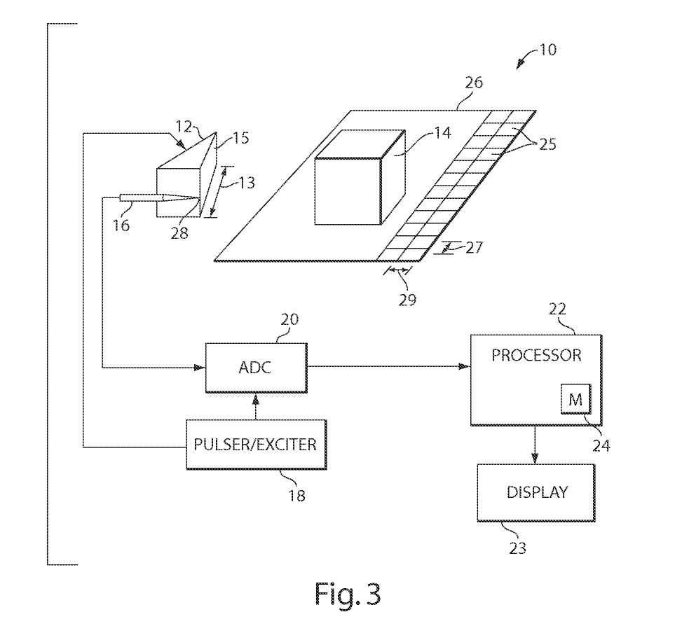

Referring to FIG. 3, a two-dimensional ultrasound imaging system 10 includes a transducer (transmitter) 12, an object under test 14, a receiver 16, a pulser/exciter 18, and analog-to-digital converter (ADC) 20, a processor 22, and a display 23. The pulser 18 is connected to the transducer 12 and the ADC 20. The receiver 16 is connected to the ADC 20, which is connected to the processor 22. The processor 22 is configured to process incoming information to construct two-dimensional images of the object under test 14. The processor 22 is preferably a computer with memory 24 that stores computer program code instructions and a central processing unit configured to read the code and perform functions in accordance with the code to construct images. The object 14 can be any item amenable to imaging with ultrasound, e.g., a person, an animal, structural materials, underwater objects, underground objects, intracranial objects, deep-tissue objects, breast objects (e.g., calcifications), and many others.

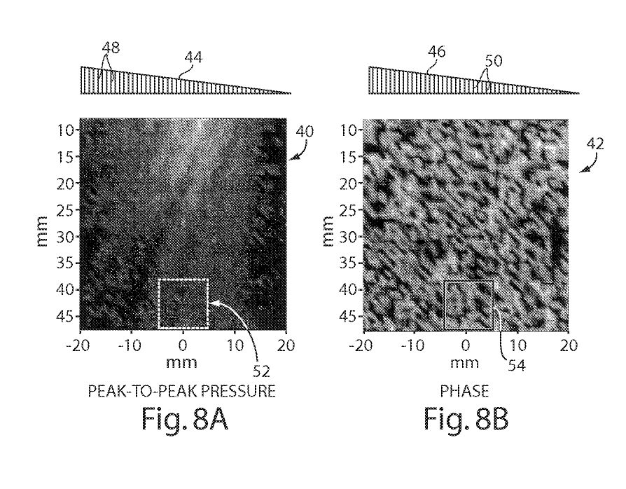

The transducer 12 is configured to provide a complex radiation field with a frequency of signal emitted by the transducer 12 varying along its length. Here, the transducer 12 is an emitter made of a piezo ceramic or a composite containing a piezo ceramic such as lead-zirconate-titanate (e.g., PZT-4 single crystal) and has a hexahedral right prism (or “doorstop”) shape. The transducer 12 provides a frequency response that varies linearly along a length 13 of the transducer's aperture 15. The length 13 of the aperture 15 is preferably at least about three times (or at least about five times, or at least about 10 times, or at least about 20 times) a wavelength of a highest frequency produced by the transducer 12. The transducer 12 produces a divergent broadband beam with a complex field profile such that each pixel 25 in an ROI 26 receives a unique waveform as a function of time from inducement of the signal (see FIGS. 1 and 8). The pixels 25 have center-to-center spacings (i.e., pitches) 27, 29 in rows and columns of about ⅛ of the wavelength of the center imaging frequency produced by the transducer 12. Here the pitch is about 1 mm. The ultrasound field is such that at no two pixels 25 in the ROI 26 receive identical waveforms. The waveforms received differ in shape and/or timing. For example, referring to FIG. 1, for a pulse input to the transducer 12, the waveform received at a first pixel 25 may have a non-zero portion of the shape and timing shown in FIG. 1B while the waveform at a second, different pixel will have a waveform with a non-zero portion having a different shape than the waveform shown in FIG. 1B, a different timing relative to time zero, or both. Preferably, the transducer 12 is air-backed to help provide good Quality Factor (Q) at a giving point on the transducer 12. The transducer 12 is configured to produce signals of frequencies over a large bandwidth from a relatively low bandwidth compared to previous ultrasound imaging systems to a high-end frequency, e.g., from about 200 KHz to about 6.5 MHz, i.e., about 33:1).

The exciter 18 is configured to provide excitation signals to the transducer 12, preferably to produce a broadband, dispersive signal. The exciter 18 is configured to provide an impulsive signal, preferably with a programmable amplitude, with a time duration much shorter (e.g., less than 0.1 times) than that of a period of the highest radiating frequency produced by the transducer 12. The exciter 18 preferably provides the impulsive signal with a repetition frequency that is equal to or lower than that of the lowest radiating frequency produced by the transducer 12. The exciter 18 is further configured to provide timing information to the ADC 20 indicative of when impulses are provided to the transducer 12 to produce ultrasound waves. An exemplary exciter 18 is pulser-receiver Model 500PR made by Panametrics, Inc. of Waltham, Mass.

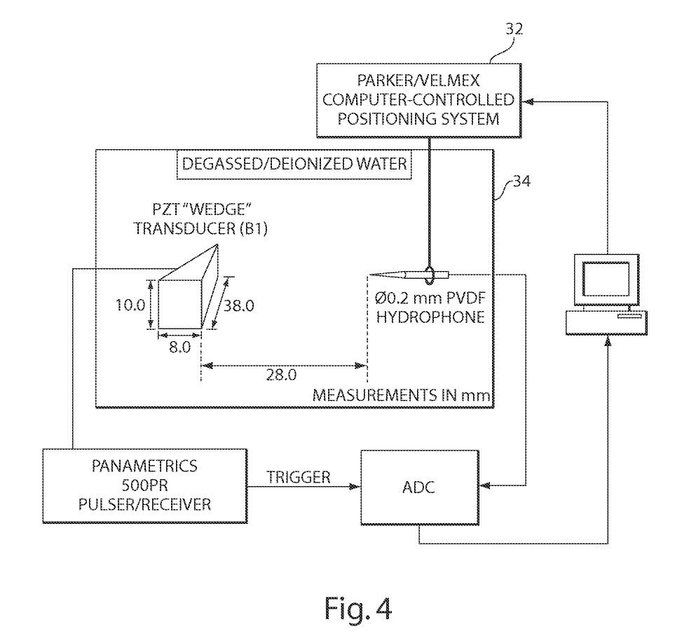

The receiver 16 is configured to receive signals from the transducer 12 that are reflected by the object 14 and to transmit indicia of the received signals. The receiver 16 can take a variety of forms and here is a needle-shaped probe hydrophone with a polyvinyldiflouride (PVDF) tip 28. The probe 16 is disposed in the transducer 12, e.g., by insertion into a hole formed in the transducer 12. The probe 16 is configured to transduce reflected signals received by the probe 16 into analog signals indicative of the received reflected signals and to transmit the transduced signals to the ADC 20. Referring also to FIG. 4, using a calibration setup 11 of the system 10, the receiver 16 can be moved through the ROI 26 by a positioner 32 in a tank 34 containing degassed/deionized water, under control of the processor 22, in the absence of the object 14 to map the signals throughout the ROI 26. Many different positioners 32 may be used, such as bi-directional, motor-driven positioners made by Velmex, Inc. of Bloomfield, N.Y. or Parker Hannifin of Cleveland, Ohio. The calibration setup 11 can be used to provide calibration information for cross-correlation by the processor 22 of the calibration information and signals received during use with the object 14. Various hydrophones may be used as the receiver 16 such as a 0.2 mm PVDF hydrophone produced by Precision Acoustics of Dorchester, UK.

The ADC 20 is configured to convert analog information from the receiver 16 and the exciter 18 into digital form. The ADC 20 converts analog representations of received reflected signals as provided by the receiver 16 and provides digital reflected-signal indicia to the processor 22. The ADC 20 also converts analog signals from the exciter 18 regarding timing of pulses sent to the transducer 12 and transmits digital signals regarding this timing information to the processor 22.

The processor 22 is configured to analyze information from the ADC 20 to reconstruct two-dimensional images of the object 14. The processor 22 memory 24 stores calibration information regarding the field in the ROI 26. The processor 22 is configured to analyxe information regarding the timing of pulses sent by the exciter 18 and information regarding signals reflected by the object 14 and received by the receiver 16. The processor 22 can analyze the timing information and the reflected-signal information and cross-correlate with the calibration information to determine reflections due to a pixel in the ROI 26. Referring to FIG. 1C, the processor 22 can cross-correlate a particular calibration signal with the received signals in accordance with Eq. (11), or other inversion methods as appropriate, and determine that a signal from the pixel corresponding to particular calibration signal exists in the received signals if a spike occurs in a cross-correlation plot 30 at time t=0. If a reflected signal is present from the particular pixel, then the processor can determine a pixel intensity for that pixel in an image of the ROI 26 by comparing the intensity of the received reflected signal from that pixel and the intensity of the calibration signal at that pixel. The processor 22 can analyze the timing information and the reflected-signal information and cross-correlate with the calibration information for each pixel in the ROI 26 and determine pixel intensities for the ROI 26 and provide these pixel intensities to the display 23.

The display 23 is configured to produce an image using the determined intensities for the pixels in the ROI 26. The display 23 uses the pixel intensities and corresponding indicia of the pixel locations received from the processor 22 to map the intensities to an image and display the image in accordance with the intensities.

### Operation

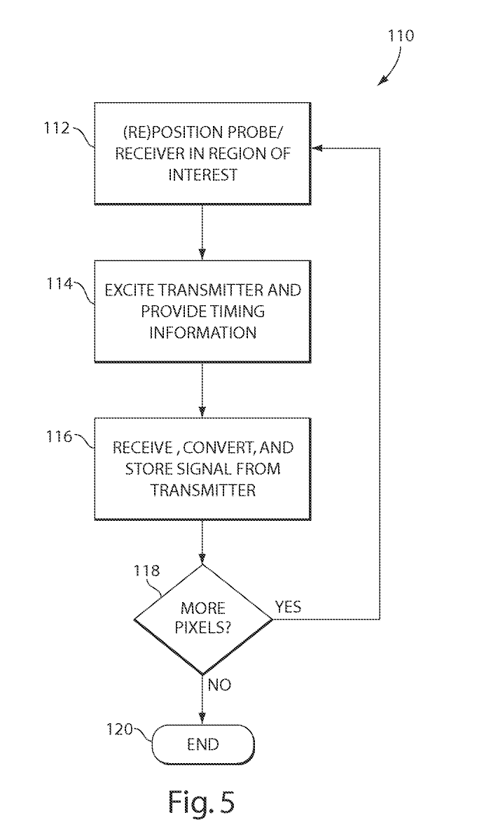

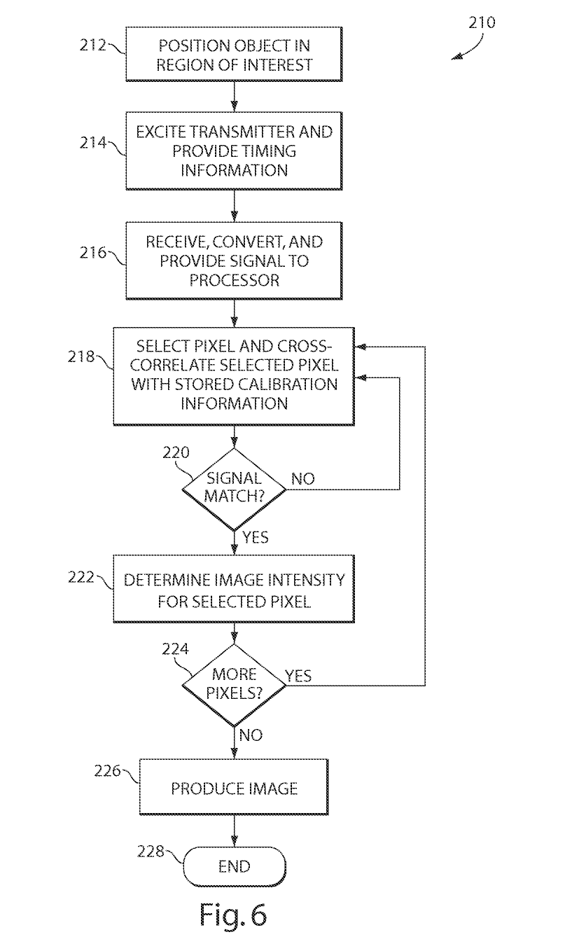

In operation, referring to FIG. 5, with further reference to FIGS. 3-4, a process 110 for producing calibrating the system 10 includes the stages shown. The process 110, however, is exemplary only and not limiting. The process 110 may be altered, e.g., by having stages added, removed, or rearranged.

At stage 112, the receiver 16 is positioned within the ROI 26. The receiver 16 is preferably stepped through each pixel in the ROI 26, e.g, by rows of pixels, by the positioner 32 under control of the processor 22.

At stage 114, the transmitter 12 is excited and timing information is provided to the processor 22. The exciter 18 sends a sequence of pulses to the transducer 12 which each excites the transducer 12, causing the transducer 12 to produce a complex ulstrasound waveform in the ROI 26. The exciter 18 also provides information to the ADC 20 indicative of the timing of each of the pulses. The ADC 20 converts the signals from the exciter 18 to digital format and provides the digital timing information to the processor 22.

At stage 116, signals from the transmitter 12 are received, converted to digital, and stored. The signals transmitted from the transducer 12 are received by the probe 16. The probe 16 transduces the signals into analog electric signals and sends these signals to the ADC 20. The ADC 20 converts the analog signals from the receiver 16 to digital signals and sends the digital signals to the processor 22. The processor 22 stores the signals from the ADC 20 in the memory 24.

At stage 118, an inquiry is made as to whether there are more pixels for which the field signal should be recorded. The processor 22 determines whether all pixels in the ROI 26 have been visited by the receiver 16 and had the corresponding signal from the transmitter 12 received and stored. If not, then the process 110 returns to stage 112 where the receiver 16 is repositioned by the positioner 32. If there are no more pixels in the ROI 26 to calibrate, then the process 110 ends at stage 120.

In operation, referring to FIG. 4, with further reference to FIG. 2, a process 210 for producing a two-dimensional image using the system 10 includes the stages shown. The process 210, however, is exemplary only and not limiting. The process 210 may be altered, e.g., by having stages added, removed, or rearranged.

At stage 212, the object 14 is positioned within the ROI 26. The object 14 is preferably approximately centered in the ROI 26 or otherwise positioned such that the entire object 14, or at least the portion(s) of interest is(are) within the ROI 26. The object 14 is preferably held stationary during other stages of the process 210.

At stage 214, the transmitter 12 is excited and timing information is provided to the processor 22. The exciter 18 sends a sequence of pulses to the transducer 12 which each excites the transducer 12, causing the transducer 12 to produce a complex ultrasound waveform in the ROI 26. The exciter 18 also provides information to the ADC 20 indicative of the timing of each of the pulses. The ADC 20 converts the signals from the exciter 18 to digital format and provides the digital timing information to the processor 22.

At stage 216, signals from the transmitter 12 are reflected, received, converted to digital, and sent to the processor 22. The signals transmitted from the transducer 12 are reflected by portions of the object under test 14 and received by the probe 16. The probe 16 transduces the signals into analog electric signals and sends these signals to the ADC 20. The ADC 20 converts the analog signals from the receiver 16 to digital signals and sends the digital signals to the processor 22.

At stage 218, the pixels in the ROI 26 are analyzed to determine whether signals are reflected from the object 14 corresponding to the pixel location. The processor 22 selects a pixel and retrieves its calibration information from the memory 24. The processor 22 cross-correlates the retrieved information with the signals received by the receiver 16 and analyzes the cross-correlation result.

At stage 220, the processor 22 determines whether the received signals include a signal reflected from the portion of the object 14 corresponding to the selected pixel. If the cross-correlation indicates that a portion of the received signals corresponds to the selected pixel, e.g., by a spike being present in the plot 30 at time t=0, then the process 210 proceeds to stage 220. Otherwise, the process 210 returns to stage 218 where another pixel is selected and its calibration information retrieved.

At stage 220, the processor 22 determines an intensity of the pixel for display as part of an image. The processor 22 analyzes the strength of the reflected signal corresponding to the selected pixel, e.g., relative to the calibration intensity. Based on this analysis, the processor 22 determines a pixel intensity for an image, and provides the intensity and an indication of a corresponding pixel location to the display 23 and/or stores this intensity information for aggregation with intensity information for other pixels for producing an image from the pixels in the ROI 26.

At stage 224, an inquiry is made as to whether there are more pixels for which it should be determined whether a reflected signal is received. The processor 22 determines whether all pixels in the ROI 26 have been analyzed for the presence of a reflected signal. If not, then the process 210 returns to stage 218 where the processor 22 selects another pixel and retrieves its calibration information. If there are no more pixels in the ROI 26 to analyze, then the process 210 proceeds to stage 226 where the display 23 aggregates the pixel intensity and location information, or uses aggregated information supplied by the processor 22, to produce an image of the ROI 26, and the process 210.ends at stage 228.

### Simulations/Experiments

Simulations and experiments have confirmed that cross-correlation may be performed to provide the desired inversion using the field produced by the transducer 12, with the transducer 12 radiating a desired overall transducer bandwidth, and desired variation in the complex pressure field as a function of frequency. It is desired that the pixel size/pitch is at least as small as a desired size, where pixel size dx is given by: dx=c*IFT (abs (spectrum)), where c is the speed of sound. IFT( ) is the inverse Fourier transform, abs indicates absolute value, and spectrum is the frequency response of the transducer. Two-dimensional images are assembled by correlating the time history of the received signal with the known response for a scatter at each location in the ROI 26.

Simulation

Simulation of the acoustic pressure at the receiver was performed using a discrete approximation to the Rayleigh-Sommerfeld integral:

$${p}_{R} ( t ) = {\sum }_{Q} {\sum }_{S} [ {q}_{κ} \frac{{ⅇ}^{ⅈ {k}_{s} {r}_{s}}}{{r}_{s}} + {q}_{κ} \frac{{ⅇ}^{ⅈ {k}_{s} {r}_{s}}}{{r}_{s}} ] \frac{{ⅇ}^{ⅈ {k}_{s} {r}_{R}}}{{r}_{R}} {ⅇ}^{ⅈ {ω}_{s} t} Δ S Δ R , \qquad ( 12 )$$

where S is a section of the surface area of the transducer and R is the scattering cross section of the scattered field. The transmission transducer face was situated in the Cartesian y-z plane, symmetric about the y-axis. In the simulations, the emitting transducer length was situated along the y-axis, while the receiver 16 was point-like and could be located at arbitrary points in space. The resonance frequencies varied linearly along the length of the radiating face. For the calculation of Eq. (12), the transducer surface was divided into squares with dimensions equal to (¼-wavelength)² of the highest frequency for a given transducer 12.

Image construction was performed as described above. This process is illustrated conceptually in FIG. 1, with a signal at the receiver 16 and a trial signal, indicating the unique, or nearly unique, waveform produced by scattering from a specific point in the ROI 26. Continuously-driven transducers were modeled with linearly varying frequencies. The ROI 26 contained a planar scattering field q_{κ}(r), situated in the imaging plane.

Transducers

Two prototype transducers 12 were constructed and used for this study: one was cut from a 25 mm×15 mm×4.4 mm (length×width×thickness) PZT-4 crystal (Transducer A), and the other was cut from a 38 mm×10 mm×8.0 mm (length×width×thickness) PZT-4 crystal (Transducer B). The transducers 12 were all electrically poled to operate in their thickness modes, at their fundamental frequencies of 0.5 MHz (Transducer A) and 0.25 MHz (Transducer B). In each case, the crystals were cut diagonally through the thickness dimension using a diamond-wire saw. The cut crystals were mounted in machined acrylic housings such that they were air-backed with their radiating surfaces electrically grounded. Two layers of conductive epoxy (e.g., Metaduct 1201 made by Mereco of West Warwick, R.I., USA) were applied to the cut surfaces and wired as the actuation electrode.

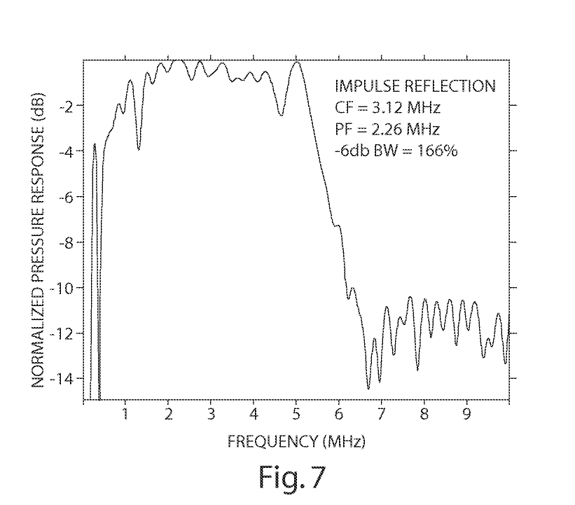

The impulse response of Transducer A was measured by analyzing the results of an impulse-reflection signal. The transducer 12 was actuated by broadband spike excitation using a Panametrics exciter Model 500PR and the signal was reflected from a submerged planar steel target oriented perpendicular to the transducer's axis of propagation. The reflected time signal, as received by the same transducer, was Fourier transformed to yield a frequency response of Transducer A (FIG. 7). The −6 dB bandwidth (BW) was measured to be 166%, with a center frequency (CF) of 3.12 MHz and a peak frequency (PF) of 2.26 MHz.

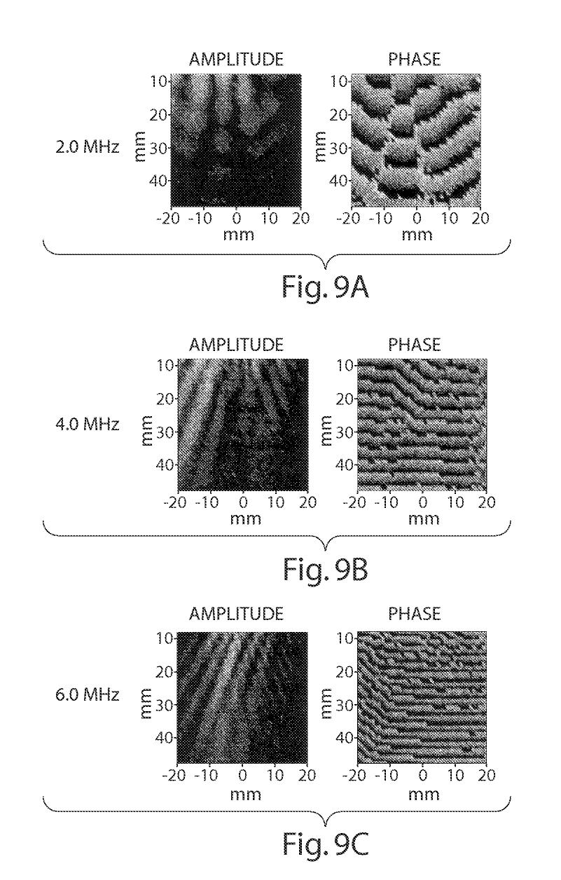

The radiation field frequency content of Transducer B was experimentally determined via three methods within the ROI 26. First, a two-dimensional pressure scan of the radiated field was performed with a 0.2-mm diameter PVDF probe 16 (FIG. 4). As the transducer 12 was actuated by broadband spike excitation, the pressure probe 16, in conjunction with the computer-controlled positioner 32 (made by Parker Hannifin) and an oscilloscope system (made by Tektronix of Beaverton, Oreg., USA, model TDS380), recorded a 20-μs sequence of measured pressure for each point in a spatial Cartesian grid. The peak-to-peak maximum pressure and relative phase for a 40 mm×40 mm plane (1 mm measurement resolution) centered 28 mm from the face of the transducer 12 and oriented parallel to the length dimension are shown in plots 40, 42 in FIGS. 8A and 8B, respectively. The position and orientation of the transducer 12 are indicated by wedge-shaped blocks 40, 42 above the plots. Vertical hash marks 58, 50 within these blocks 40, 42 are parallel to the piezoelectric polarization direction of the PZT crystal. Dashed and solid boxes 52, 54 set within the radiated fields delineate the ROI 26 for experiments performed with this transducer 12. In addition, using Matlab® (made by Mathworks of Natick, Mass., USA), these measured fields were separated in frequency to demonstrate the varied spatial signature that are created different frequencies. Results for the 2.0 MHz, 4.0 MHz, and 6.0 MHz cases are shown in FIGS. 9A, 9B, and 9C, respectively.

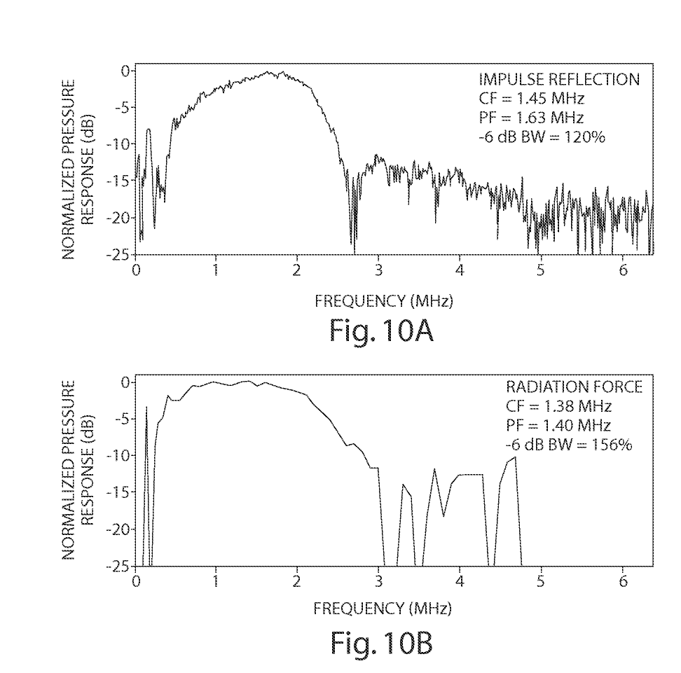

A second method to characterize the transducer 12 was based on an impulse send-receive technique in which a spiked excitation would be propagated and reflected from a near-perfect reflector. The reflected signal would then be received by the same transducer 12 and analyzed. With a setup similar to that shown in FIG. 4, Transducer B was actuated with a spike, the resulting pressure wave was propagated 14 mm to a planar water-air interface, and then the reflected signal received by the same transducer 12. The time signal was analyzed to yield a frequency-dependent pressure response as shown in FIG. 10A. With this method, the −6 dB bandwidth was measured to be 120%, with a center frequency of 1.45 MHz and a peak frequency of 1.63 MHz.

A third measurement of the frequency response of Transducer B was based upon a radiation force effect, in which the force exerted by a propagating ultrasound wave onto a perfectly absorbing target is in direct proportion to the impinging acoustic energy. The transducer 12 was positioned to direct its beam into an absorbing target, which was coupled to a digital force balance (made by Mettler Toledo of Columbus, Ohio, USA, model PR2003DR). The output power of the transducer 12, driven with continuous-wave actuation, was measured from 0.1 MHz to 7.0 MHz. The results are plotted in FIG. 10B. The radiation force measurement yielded a −6 dB bandwidth of 156%, with a center frequency of 1.38 MHz and a peak frequency of 1.40 MHz.

Experiments

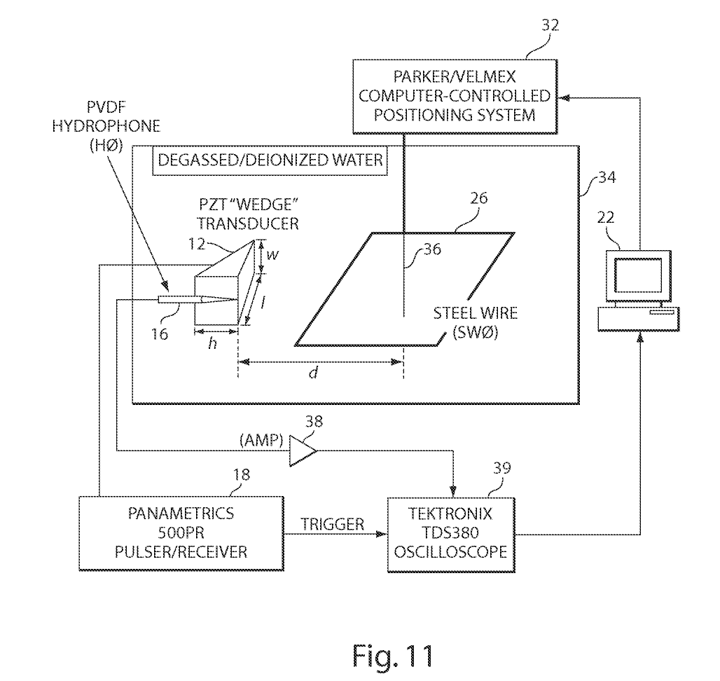

For the first experiment, Transducer A was mounted in a rubber-padded tank 34 filled with deionized water (FIG. 11). A 0.5-mm diameter polyvinylidene flouride (PVDF) hydrophone (made by Precision Acoustics of Dorchester, UK) situated next to the transducer 12 served as the receiver 16. To obtain scattering P_{Q}(r_{R}, t) as a function of position, a vertically-oriented 0.2-mm diameter steel wire 36 was guided to arbitrary positions in the tank 34 using a stepper-motor-controlled 3D positioning system 32 (made by Velmex of Bloomfield, N.Y., USA). The response of the hydrophone 16 was sent through an amplifier 38 and recorded by an oscilloscope 39 (made by Tektronix® of Beaverton, Oreg., USA). The wire positioning and data acquisition were both computer controlled. The wire 46 was scanned over a 30 mm×20 mm area 26 in front of the transducer 12, with waveforms from the hydrophone 16 recorded at 0.2-mm intervals. The center of this scanned area was located approximately 15 mm in front of the transducer 12. Following this measurement, objects were placed in front of the transducer 12 and the waveforms were again recorded. This set of single waveforms was processed by calculating the cross-correlation presented in Eq. (11) for each point in the ROI 26 scanned with the wire 36.

The second experiment explored the proposed imaging modality with a lowered center frequency of field excitation and a smaller scattering target. Transducer B was positioned in the experimental setup as diagrammed in FIG. 11. A 0.13 mm diameter steel wire 36, which was coupled to and positioned by a stepper-motor-controlled positioning system 32, was guided throughout an ROI 26 with relatively high sensitivity (FIG. 9). For each location of the wire 36, which was sequentially positioned in Cartesian coordinate positions throughout a 10 mm×10 mm planar area 26 in 0.5-mm increments, a 20-μs time sequence of the received signal including the reflection from the wire target 36 was recorded. The scanned field 26 was centered 45 mm from the face of the transducer 12. This scan was repeated twice for the same field 26, and the results were averaged at each spatial location to reduce noise artifacts. Next, steel wire targets 36 were placed in various locations within the ROI 26 and a single 20-μs time sequence of each reflected signal was recorded. Each of the waveforms, corresponding to various placements of scattering sites, was sequentially cross-correlated according to Eq. (11) with each saved waveform from the composite scans of the field. The results of the cross-correlations were analyzed to determine the accuracy in reproduction of the scattered field.

### Simulation/Experiment Results

Simulation

Simulations were performed to calculate the pressure field and received signal from a 40 mm×10 mm planar transducer with linear frequency variation between 0.3 MHz and 2.5 MHz over its length. A point-receiver with a flat frequency response over relevant range was situated at the low frequency end of the transducer 12, and centered about its width. Both the surface dimensions and frequency range were selected to approximate the radiation behavior of Transducer B. An ROI 26 was selected in an area in front of the transducer 12 between the distances of 10 mm to 30 mm normal to the transducer surface and −5 mm to 35 mm along its length.

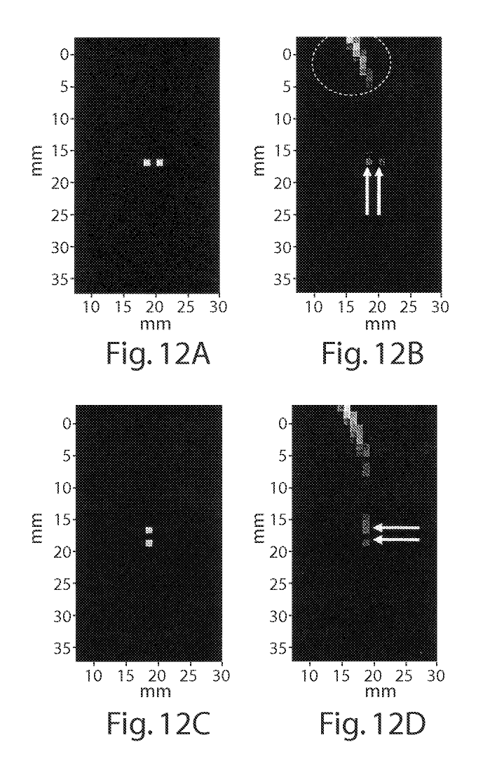

Simulated results verified the ability of the method and selected geometry to detect and localize one or more scatterers within the ROI 26 under idealized conditions. All scatterers in the ROI were assigned a scattering strength of q_{κ}=0.1, caused by a speed of sound increase from 1.50×10³ ms⁻¹ to 1.58×10³ ms⁻¹. With the selected geometric and frequency configuration, localization was evident in both the axial and radial direction of a 0.25 mm diameter object at or below the range of wavelengths in the signal (0.60 mm to 5.0 mm). This indicated the ability to image sub-wavelength scatterers without the characteristic blurring associated with backscattered ultrasound detection. Detecting the separation between two or more 0.25 mm-diameter scatterers placed 1 mm or more apart was also possible with the low frequency emitter 12. In a series of images that moved to objects 14 successively further from each other (simulated at 1 mm intervals), a 1 mm separation was detectable along the normal axis (FIG. 12B), while 2 mm separation could be detected along the transducer length (FIG. 12D). In both of these simulations, as well as subsequent simulations with multiple objects 14, reconstruction artifacts appeared in the images, as indicated by the dotted outlines in FIGS. 12B and 12D, with arrows indicating the objects.

Experimental

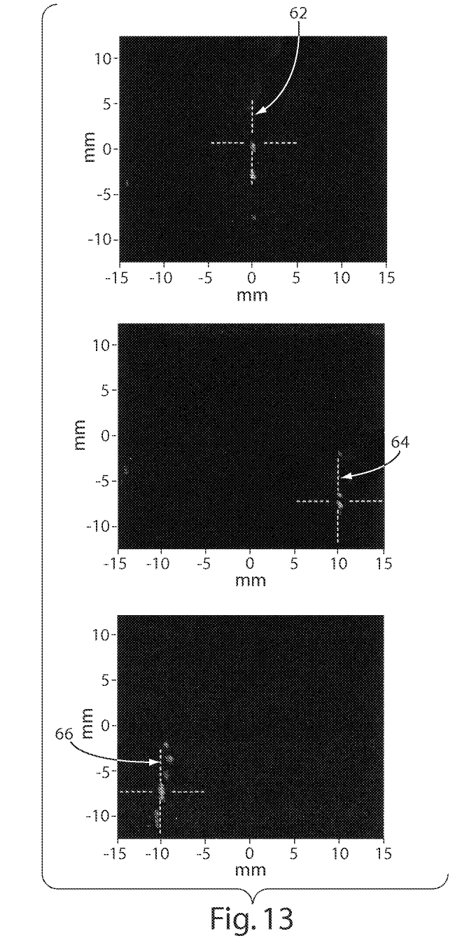

Three examples of the resulting measured field analyses are presented in FIG. 13 for scans performed with Transducer A. The intersections of dashed crosshairs 62, 64, 66 indicate the actual positions of scatterers. Relative to the images, the transducer 12 was situated to the left, with the thick portion towards the bottom. The cross-correlation analyses of these scans yielded high correlations at the field location corresponding to the locations of scatterers, marked by the intersection of the dashed crosshairs in FIGS. 13A, 13B, 13C. The grayscale intensities in these images were set to be proportional to the degree of correlation at the origin of the cross-correlation analysis, as diagramed in FIG. 1. In addition, a linear interpolation filter was applied between adjacent pixels. In each scan, there was evidence of correlation artifacts, predominantly in the radial direction of the images (i.e., orthogonal to the propagation axis of the transducer 12).

Similar scans were performed using Transducer B to, among other things, examine the feasibility of using an excitation source of substantially lower center frequency (compared to previous systems of similar resolution) to localize scatterers of reduced geometry. Within the ROI 26 for these scans (FIG. 9), it can be seen that the complex pressure field spatial distribution is asymmetric and irregular, not only in magnitude, but also in phase. This asymmetry helps assure that the complex backscatter at each point within an ROI 26 will have a unique value. The scatterers in this case were steel wires with measured diameters of 0.13 mm, and the excitation center frequency had a wavelength in water of 1.1 mm (calculated using 1.38 MHz, the center frequency as measured by the radiation force method and a sound speed in water of 1500 ms⁻¹).

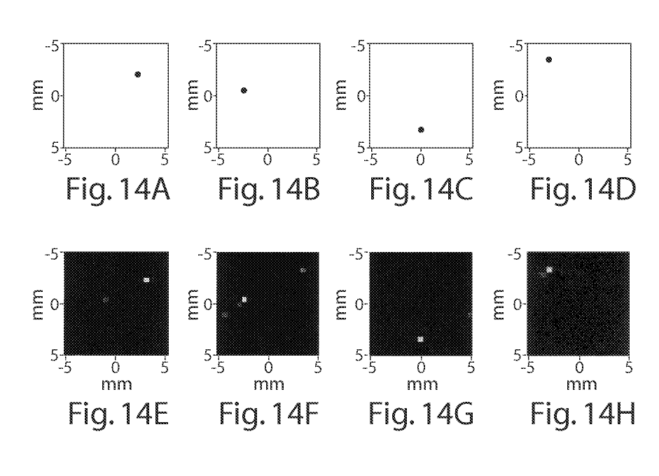

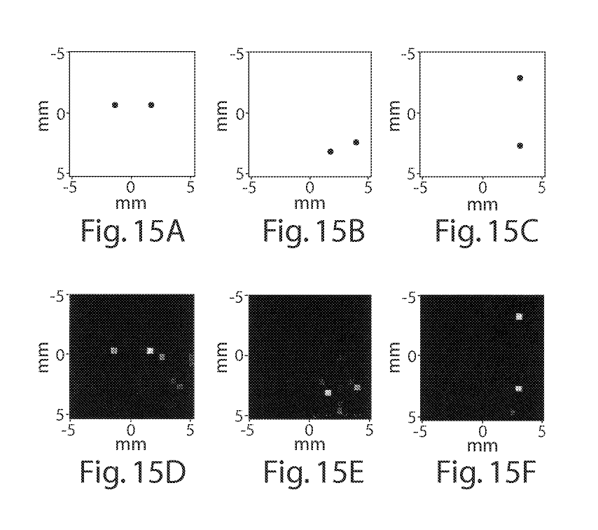

Four examples of analyzed scatter fields for single targets are shown in FIG. 14. FIGS. 14A-14D indicate the actual positions of the scatterers in the ROI 26 and the corresponding field reconstructions are shown directly below in FIGS. 14E-14H. In these images, the source transducer 12 is situated to the bottom of the diagram with the thick portion (fₘᵢₙ) towards the right, and the receive hydrophone 16 is situated 10 mm to the right of the excitation transducer 12. The results of the cross-correlation analyses show a spatial correlation between the measured and actual scatter site to within 0.5 mm for single scatterers. In FIG. 15, three examples are shown for a series of field reconstructions for single excitation of a field with simultaneous two point scatterers, each with a diameter of 0.13 mm. FIGS. 15A-15C indicate the actual positions of the scatterers in the ROI 26 and the corresponding field reconstructions are shown directly below in FIGS. 15D-15F. These measurements were performed with the same experimental setup and reconstruction algorithm as those of FIG. 14. The localization of two distinct scatterers is to within 0.5 mm and although there are artifacts, the contrast is sufficient to identify the scatter sites.

### Discussion

The experiments were devised to determine whether a single sonicate-receive sequence can be used to localize backscatter signals not only along the ultrasound axis of propagation, but also along the radial direction. Initial simulations suggested that it is possible depending upon the bandwidth of the sonicating transducer. The simulation revealed that even relatively low bandwidths were able to detect and localize small point-like objects. Based on numeric results, transducers with varied excitation and reception parameters were constructed to test in an experimental setup. These experiments supported the simulated cases.

For the first experiment, in which Transducer A was the field excitation source 12, and a needle hydrophone was the receiver 16, some artifacts appeared in the images. The underlying assumption of a point receiver is that its surface area is much small than the received wavelengths, which was not the case for Transducer A. Using Transducer A, however, two-dimensional localization of scattered objects was performed, albeit with the introduction of interference artifacts in the images. The predominance of artifacts in the radial directions, rather than the axial directions, implied that the temporal isolation of the backscattered signal gives a higher localizing value than the spectral signature.

Transducer B was designed to have a lower center frequency. The lower center frequency of operation of Transducer B was desired to help ascertain the possibility of sub-millimeter localization resolution in distal regions of an attenuating image field.

A transducer geometry with a larger thickness dimension, and tapered linearly to zero, was hypothesized to give a lower center frequency one with a smaller thickness dimension. In this study, Transducer B had a thickness that was nearly twice that of Transducer A. The characterization of Transducer B confirmed a lower center frequency (1.67 MHz lower than Transducer A). This parameter comparison between Transducer A and Transducer B was made using the parameters as obtained from impulse reflections. The radiation force measurements performed with Transducer B yielded a 0.07 MHz lower center frequency than that of the impulse reflection measurement. With each measurement method, however, Transducer B was shown to operate at a lower center frequency than Transducer A. The bandwidth of Transducer B was determined to be about 120%.

The results of image reconstructions from scans performed with Transducer B were able to address the issue of sub-millimeter localization in distal regions. The results also confirmed the existence of radial artifacts for transducer bandwidths between 120% and 166% and center excitation frequencies of 1.38 MHz to 3.12 MHz. The data demonstrated that spatial localization resolutions substantially lower than the excitation wavelengths can be expected even when the wavelength of the center excitation frequency is an order-of-magnitude larger than the scattering targets' spatial dimensions. Even for the highest-efficiently radiating frequency (here 2.45 MHz) as determined by the transducer in FIG. 10A, the corresponding wavelength is more than 4 times that of the scattering profile of the scatterers used. It was also demonstrated that receiver size, relative to wavelength, does not present a significant impediment to this localization method. In this case, compared qualitatively with the results of experiments performed with Transducer A, signal integration across a larger receiver diaphragm had a negligible effect on the preservation of a unique backscatter signature. When more than one scatterer was introduced into the ROI, accurate localization was also achieved.

### OTHER EMBODIMENTS

Other embodiments are within the scope and spirit of the invention. For example, due to the nature of software, functions described above can be implemented using software, hardware, firmware, hardwiring, or combinations of any of these. Features implementing functions may also be physically located at various positions, including being distributed such that portions of functions are implemented at different physical locations. Further, the system 10 can be used to provide three-dimensional images. Also, while the exciter 18 discussed above is configured to provide electrical excitation signals, other forms of excitation signals may be provided such magneto-restrictive excitation signals for underwater applications.

A “signal” or “stream” may be modified by a component and referred to herein (in the description and/or claims) as “the signal” or “the stream” both before and after the modification. For example, a “stream” or “signal” that is received by the receiver 16 can be converted to electrical format and can be modified by other components (e.g., the ADC 20, the processor 22) and still be referred to as “the stream” or “the signal” before and after the receiver and the other components.

Further, while the description above refers to “the invention,” more than one invention may be disclosed.

What is claimed is:

1. An ultrasound imaging system for use in producing an image of an object in a region of interest, the system comprising:
an exciter configured to provide an excitation signal;
a transducer coupled to the exciter and configured to produce, in response to the excitation signal, an ultrasound field whose complex frequency content varies with field location;
a receiver configured to receive ultrasound signals reflected by the object and to produce indicia of the received reflected ultrasound signals; and
a processor coupled to the receiver and configured to cross-correlate the indicia of the received reflected ultrasound signals with indicia of the ultrasound field at pixels in the region of interest to determine image pixel intensities of the region of interest for producing an image.

2. The system of claim 1 wherein the transducer is configured to produce the ultrasound field such that the field has unique waveforms at each pixel location in the region of interest in the absence of the object, the waveforms being different in at least one of shape and timing relative to production of the ultrasound field.

3. The system of claim 2 wherein the pixels have a pitch of at least about ⅛ of a wavelength of a center frequency of the transducer.

4. The system of claim 1 the transducer is configured to provide a frequency response that varies linearly along a length of an aperture of the transducer.

5. The system of claim 1 wherein the transducer and the receiver are each stationary relative to the object and provide a single imaging channel.

6. The system of claim 1 wherein the transducer is configured as a hexahedral right prism having two nonparallel surfaces, with one of the nonparallel surfaces being a radiating surface.

7. The system of 6 wherein the transducer is polarized normal to the radiating surface.

8. The system of claim 1 wherein the excitation signal is a spike.

9. The system of claim 1 wherein the receiver is separate from, and disposed in, the transducer.

10. The system of claim 9 wherein the receiver is configured as a point receiver.

11. The system of claim 1 wherein the transducer is configured to produce ultrasound signals with frequencies from about 200 KHz to at least about 2.5 MHz.

12. The system of claim 1 wherein the transducer is configured to produce ultrasound signals over a range of frequencies with a −6 dB bandwidth of between about 120% and about 166%.

13. The system of claim 1 further comprising a display coupled to the processor, wherein the processor and the display are configured to produce a two-dimensional image of the region of interest from the image pixel intensities of the region of interest.

14. A method of imaging an object in a region of interest using ultrasound, the method comprising:
producing an ultrasound field such that waveforms at centers of predetermined pixel locations in the region of interest in the absence of the object would be unique;
receiving ultrasound signals reflected by the object;
producing indicia of the received reflected ultrasound signals;
cross-correlating the indicia of the received reflected ultrasound signals with indicia of the waveforms at pixels in the region of interest to determine image pixel intensities of the region of interest for producing an image; and
producing an image of the object using the image pixel intensities.

15. The method of claim 14 wherein waveforms at different pixels are different in at least one of shape and timing relative to production of the ultrasound field.

16. The method of claim 14 wherein producing the ultrasound field comprises providing a frequency response at a transducer that varies linearly along a length of an aperture of the transducer.

17. The method of claim 14 wherein producing the ultrasound field is performed at a transducer that is stationary relative to the object and wherein receiving ultrasound signals reflected by the object is performed at a receiver that is stationary relative to the object.

18. The method of claim 14 wherein producing the ultrasound field comprises applying a spike excitation signal to a transducer.

19. The method of claim 14 wherein producing the ultrasound field comprises producing ultrasound signals with frequencies from about 200 KHz to at least about 2.5 MHz.

20. The method of claim 14 wherein producing the ultrasound field comprises producing ultrasound signals over a range of frequencies with a −6 dB bandwidth of between about 120% and about 166%.

21. An ultrasound transducer system comprising an air-backed hexahedral right prism transducer having first and second surfaces that are nonparallel with respect to each other, the transducer being configured to receive an excitation signal and to radiate, in response to the excitation signal, ultrasound waves from the first surface, the transducer being configured to radiate ultrasound waves along a length of the first surface and having frequencies in a range from a first frequency to a second frequency, the second frequency being higher than the first frequency, and wherein the length of the first surface is at least about three times as long as a wavelength of the second frequency.

22. The system of claim 21 wherein the transducer comprises a piezo ceramic material.

23. The system of claim 22 wherein the transducer comprises a composite material containing the piezo ceramic material.

24. The system of claim 21 wherein the length of the first surface is at least about five times as long as the wavelength of the second frequency.

25. The system of claim 24 wherein the length of the first surface is at least about ten times as long as the wavelength of the second frequency.

26. The system of claim 25 wherein the length of the first surface is at least about twenty times as long as the wavelength of the second frequency.

27. The system of claim 21 further comprising an exciter coupled to the transducer and configured to provide the excitation signal to the transducer, the excitation signal comprising a broadband spike.

28. An ultrasound transducer system comprising a single transducer configured to receive an excitation signal and to radiate, in response to the excitation signal, ultrasound waves along a length of an aperture with the ultrasound waves having frequencies in a range from a first frequency to a second frequency, the second frequency being higher than the first frequency, and wherein the length of the aperture is at least about three times as long as a wavelength of the second frequency.
